(19) United States
(12) Patent Application Publication
(10) Pub. No.: US 2026/0338700 A1
(43) Pub. Date: Oct. 29, 2026

(54) METHOD AND SYSTEM FOR DETECTING CATIONIC AND ANIONIC POLYMERS IN AQUEOUS SOLUTIONS

(71) Applicants: Gerald HANNA, Sherwood Park (CA); Greg GOSS, Edmonton (CA)

(72) Inventors: Gerald HANNA, Sherwood Park (CA); Greg GOSS, Edmonton (CA)

(21) Appl. No.: 19/116,227

(22) PCT Filed: Sep. 27, 2023
(86) PCT No.: PCT/CA2023/051280

§ 371 Date: Mar. 27, 2025

Related U.S. Application Data
(60) Provisional application No. 63/410,992, filed on Sep. 28, 2022.

(51) Int. Cl. G01N 21/78; G01N 21/552; G01N 31/22; G01N 33/18

(52) U.S. Cl. CPC G01N 21/78; G01N 21/554; G01N 31/22; G01N 33/1813; G01N 33/182

(57) ABSTRACT

A method and system for using citrated gold nanoparticles to detect the concentration of cationic and anionic polymers in aqueous solutions is discussed. Citrated gold nanoparticles of a known concentration are added to an aqueous solution to bind with cationic or anionic polymers that provide a colour change to the aqueous solution that provides a visual indication of the concentration of the cationic or anionic polymer.

### CROSS-REFERENCE TO RELATED APPLICATIONS

This application claims priority of United States provisional patent application serial no. 63/410992 filed 28 Sep. 2022, which is incorporated by reference into this application in its entirety.

### TECHNICAL FIELD

The present disclosure is related to the field of detecting polymers in aqueous solutions, in particular, using citrated gold nanoparticles to detect the concentration of cationic and anionic polymers in aqueous solutions.

### BACKGROUND

The growth in industrial processes and mining activities using water in processing of ore or extraction of crude petroleum has increased the total volume of water and mine wastes, resulting in the potential contamination of the environment and water resources. As a result of new regulations, a desire for increased cost efficiency, and a heightened awareness, industries and governments are adopting strategies for efficient water treatment that could enable water reuse. Coagulation-flocculation processes are among the most widely employed techniques for purification of urban and industrial wastewaters to remove fine particulate matter that does not readily settle/sediment out in settling ponds. The addition of positively charged polyelectrolyte polymers to industrial wastewater facilitates the settling and removal of negatively charged suspended particles (e.g., clays) an also has been shown to remove other contaminants of concern. Cationic polyelectrolytes are a class of polymers possessing multiple positive charges that can act as coagulants and adsorb to the surfaces of negatively charged particles. They play an essential role in removing many harmful water contaminants including suspended sediments. Among this class of cationic polymers, poly-diallyldimethylammonium chloride (“poly-DADMAC” or “p-DADMAC”) is considered to be one of the most effective and widely used polyelectrolytes in wastewater clarification. The widespread use of poly-DADMAC arises partly because of its water solubility, high charge density, high molecular weight, as well as that it does not form harmful by-products in the presence of chlorine. Despite these benefits, charged polymers such as poly-DADMAC pose a threat to fish because they interact with gills and hinder efficient oxygen transfer, with lethal concentrations measured as low as 200 ug/L (0.2 mg/L) in waters with low dissolved organic matters such as those occurring in mountain streams.

The Clearflow Group Inc. of Sherwood Park, Alberta, Canada has developed a reagent CN369™ that, when mixed in appropriate ratios with cationic polymer treated water, neutralizes the cationic polymer-mediated toxicity and sub-lethal effects on fish physiology are eliminated (Clifford et al, 2022). Despite these very promising and game changing findings, a key (and potentially application limiting) concern regarding the practical implementation of cationic polymer mitigation using CN369™ is establishing appropriate dosing protocols and refinement of the cationic polymer additions. There are currently no satisfactory rapid analytical protocols for this group of compounds (anionic and cationic polymers) at the low yet toxic concentrations relevant for regulatory requirements. The difficulty in measuring polymer concentrations, after treatment, in the field commonly results in dosing inaccuracy and potential for excessive release of unbound polymer. It is well known that below optimum polymer dosage, the coagulation and flocculation are inefficient while overdosing wastes valuable resources and increases toxicity of discharged water. Therefore, it is pertinent that an accurate measure of initial polymer concentrations in the process water prior to treatment in the field or discharge to the environment.

There are a variety of approaches for sensing or detecting cationic and anionic polyelectrolytes in “clean” water (e.g., turbidimetry/nephelometry, spectrofluorometry, spectrophotometry, viscometry, colloid titration and luminescence titration) but these techniques are time-consuming (often taking days to get results), resource intensive, require high capital cost and trained analysts, and more do not perform well with complex mixtures such as wastewater, biosolids and environmental samples.

The current methods used for neutralization of toxicity in polymers are not consistent, quantifiable nor detectable in the release streams to the environment. Neutralization, if ever done or attempted, is done with the use of a liquid or a granular chemical. This process requires monitoring and very costly equipment and access to power for the equipment operation.

Government regulators now want specific and defined, quantitative measurements for detection of residual cationic polymers and neutralization prior to release of treated water. Government regulators, such is in the province of Alberta, Canada, are now forcing change within industry to come up with methods to reduce toxicity and limit the use of toxic chemicals such as Cationic polymers. The alternative to making change within industrial process according to the regulators is to eliminate the use of cationic polymers in open industrial environments such as mining, etc. and this is not currently possible due to load factors, temperature and flow dynamics in industry.

Current technology does not allow for:
A quantified and consistent value for neutralizing cationic polymer toxicity in industrial source water.
A passive form (non-mechanical) method of treatment for neutralizing cationic toxicity in flow streams.
A method of detection of residual cationic polymers at or below 1 milligram per liter (either in lab or in the field).

Current product technology is:
pH sensitive, a variation in pH in the source water can dramatically reduce the efficacy of the product used for neutralization.
Temperature sensitive, efficacy of the current technology products can be dramatically reduced by temperature variance especially cold temperatures.
Effected by hardness levels in the source water, higher calcium and magnesium levels in the source water can greatly reduce the efficacy of the current product technology due to the ion exchange factors.

Typical dosing of polymers of all types is through:
Pneumatic process or dry feed process dosing a granular polymer into a flow stream or mixing grid system.
Liquid make-down of a granular, bead or powder polymer product.
Liquid injection of a polymer emulsion or liquid polymer mix.
The above listed process requirements need:
   Power.
   Manpower to oversee dosing—Government regulations requires up to 3 systems checks per day on liquid automated systems. This is not feasible for companies on large sites.
   The Average cost per liquid Floc dosing system is around $200,000.
   Each placement would require 2 independent liquid systems, one for Cationic and one for anionic dosing of liquids. Total investment per placement $400,000.
   Granular systems can cost $1 million dollars plus depending on volume requirements and full-time operations person plus the already existing liquid Floc system.
   Product replacement is needed constantly and in remote areas such as mines this can be very difficult due to the bulk size and access roads.
   Large industrial or mining sites would require multiple systems, one set of two systems per each flow stream. Costing is not feasible.

It is, therefore, desirable to provide a rapid field ready method and system that can detect cationic and anionic polyelectrolytes in aqueous solutions that address governmental regulations while being both cost-effective and safe for the receiving environment.

### SUMMARY

A straightforward and rapid colorimetric assay has been developed to screen for residual poly-diallyldimethylammonium chloride (“poly-DADMAC”) and any other cationic polyelectrolyte polymers in aqueous solutions using citrate-stabilized gold nanoparticles (“cit-AuNPs”) as a nanoprobe. This assay can be used directly at the worksite to detect cationic polymers in water at concentrations which could cause failure in regulatory bioassay testing (≥0.2 mg/L). The assay can also be modified to test for anionic polymers by simply altering the charge on the gold NP using amide termination of the gold nanoparticle in the detection system.

Addition of the citrate-capped 5-8 nm AuNP (gold nanoparticles) nanoprobe to an aqueous solution of cationic polyelectrolyte polymer cause aggregation of the gold nanoparticle inducing a readily observed colour change of the solution. In some embodiments, negatively charged cit-AuNPs can be used for detection of cationic polymers while positively-charged amide-terminated AuNPs can be used for detection of anionic polymers. The description below outlines the test for cationic polymers as these are the most toxic of the polymers and are most urgently needed for better environmental practice by industry. In some embodiments, the method can be configured to detect other charged anionic polymers in water by using positively-charged AuNP.

For cationic polymer testing, the change in colour is proportional to the concentration of the detected polymer at very low concentrations with a distinct visual colour change in the assay occurring between 20 and 150 μg/L of cationic polymer (in distilled water). As a result, cationic polymers can be detected in water using simple visual inspection of this colour change. Final concentrations of cationic polymer as low as 30 μg/L can be detected visually using this assay. In some embodiments, the methods and systems described herein can comprise the development of procedures to mitigate the impact of competing metal cations (e.g., Ca2+ and Mg2+) that normally complicate poly-DADMAC detection in high ionic strength solutions and the minimization of interferences by dissolved organic matter. In some embodiments, the methods and systems described herein can be used on any cationic polymer in aqueous solution.

Broadly stated, in some embodiments, a method and system is presented that can provide a straightforward, cost-effective colorimetric method that can allow for the detection of polyelectrolytes in real-time at the field site. AuNPs have seen increase applications in the detection of various analytes in the environmental, biological and pharmaceutical media. The methods and systems described herein rely on the fact that AuNPs can exhibit a characteristic and size dependant colour change arising from free electrons in the particles interacting with the incident light to cause a collective electron oscillation known as localized surface plasmon resonance (“LSPR”). In some embodiments, the methods and systems described herein can comprise this phenomenon and the fact that oppositely charged polymers and particles will assemble on mixing. In some embodiments, the polymer in the aqueous solution can interact with the AuNPs in solution and can induce aggregation resulting in a distinctive colour change that can be monitored. This can provide for a colorimetric assay for polyelectrolyte detection and measurement.

Broadly stated, in some embodiments, a method can be provided for detecting the concentration of cationic or anionic polymer in an aqueous solution where the cationic or anionic polymer has been introduced into the aqueous solution, the method comprising: preparing a first reference solution having a first colour; preparing a second reference solution having a second colour; preparing a third reference solution having a third colour; preparing a control reference solution having a fourth colour; preparing a field sample solution having a sample solution colour; and comparing the sample solution colour of the field sample solution to the first, second, third and fourth colours of the first, second, third and control reference solutions to determine the concentration of the cationic or anionic polymers in the field sample solution.

Broadly stated, in some embodiments, preparing the first reference solution can comprise: adding a chelating agent to a first vial; adding a first reference polymer solution to the first vial, the first reference polymer solution comprising a first concentration; adding distilled water to the first vial; mixing the chelating agent and the first reference polymer solution with the distilled water in the first vial; adding a reference citrate-capped gold nanoparticle (AuNP) solution to the first vial; and mixing the AuNP solution with the chelating agent and the first reference polymer solution with the distilled water in the first vial thereby producing the first reference solution having the first colour.

Broadly stated, in some embodiments, preparing the second reference solution can comprise: adding a chelating agent to a second vial; adding a first reference polymer solution to the second vial, the first reference polymer solution comprising a first concentration; adding distilled water to the second vial; mixing the chelating agent and the first reference polymer solution with the distilled water in the second vial; adding a reference citrate-capped gold nanoparticle (AuNP) solution to the second vial; and mixing the AuNP solution with the chelating agent and the first reference polymer solution with the distilled water in the second vial thereby producing the second reference solution having the second colour.

Broadly stated, in some embodiments, preparing the third reference solution can comprise: adding a chelating agent to a third vial; adding a second reference polymer solution to the third vial, the second reference polymer solution comprising a second concentration; adding distilled water to the third vial; mixing the chelating agent and the second reference polymer solution with the distilled water in the third vial; adding a reference citrate-capped gold nanoparticle (AuNP) solution to the third vial; and mixing the AuNP solution with the chelating agent and the second reference polymer solution with the distilled water in the third vial thereby producing the third reference solution having the third colour.

Broadly stated, in some embodiments, preparing the control reference solution can comprise: adding a chelating agent to a fourth vial; adding a polymer-free sample of the aqueous solution to the fourth vial; adding distilled water to the fourth vial; mixing the chelating agent and the polymer-free sample of the aqueous solution with the distilled water in the fourth vial; adding a reference citrate-capped gold nanoparticle (AuNP) solution to the fourth vial; and mixing the AuNP solution with the chelating agent and the polymer-free sample of the aqueous solution with the distilled water in the fourth vial thereby producing the control reference solution having the fourth colour.

Broadly stated, in some embodiments, the chelating agent can comprise ethylenediaminetetraacetic acid.

Broadly stated, in some embodiments, the chelating agent can comprise a concentration of about 0.1 moles/litre.

Broadly stated, in some embodiments, the first reference polymer solution can comprise a concentration of about 1 milligram/litre.

Broadly stated, in some embodiments, the second reference polymer solution can comprise a concentration of about 0.2 milligram/litre.

Broadly stated, in some embodiments, the AuNP solution can comprise a concentration of about 0.18 milligram/millilitre.

Broadly stated, in some embodiments, the polymer-free sample can be drawn from a location upstream from where the cationic or anionic polymer has been introduced into the aqueous solution.

Broadly stated, in some embodiments, a system can be provided for detecting the concentration of cationic or anionic polymer in an aqueous solution where the cationic or anionic polymer has been introduced into the aqueous solution, the system comprising: a first reference polymer solution; a second reference polymer solution; a chelating agent; a reference citrate-capped gold nanoparticle (AuNP) solution; and distilled water.

Broadly stated, in some embodiments, the system can further comprise a plurality of vials.

Broadly stated, in some embodiments, the system can further comprise a plurality of syringes for transferring fluids into the vials.

### BRIEF DESCRIPTION OF THE DRAWINGS

FIG. 1 is a diagram depicting one embodiment of colorimetric p-DADMAC/cationic polymer assay.

FIG. 2 is a photograph depicting a series of assay samples of cit-AuNPs indicating varying concentrations of p-DADMAC.

FIG. 3 is a photograph depicting four vials of differing concentrations of p-DADMAC, from left to right: 1 mg/L, 0.5 mg/L, 0.2 mg/L and 0.0 mg/L.

FIG. 4 is a photograph depicting four vials having a concentration of 1 mg/L of p-DADMAC in natural water containing different hardness and dissolved organic acid concentrations.

FIG. 5 is a photograph depicting two vials having a concentration of 0.2 mg/L of p-DADMAC in natural water containing different hardness and dissolved organic acid concentrations.

FIG. 6 is a photograph depicting two vials having a concentration of 0.0 mg/L of p-DADMAC in natural water containing different hardness and dissolved organic acid concentrations.

FIG. 7 is a photograph depicting two vials of river water with differing concentrations of p-DADMAC, from left to right: 1 mg/L, 0.5 mg/L, 0.2 mg/L and 0.0 mg/L.

FIG. 8 is a photograph depicting four vials of river water presenting colours of blue and purple/pinkish purple that indicate toxic concentrations of polymer.

### DETAILED DESCRIPTION OF EMBODIMENTS

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment can also be included in other embodiments but is not necessarily included. Thus, the present technology can include a variety of combinations and/or integrations of the embodiments described herein.

The presently disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

While the following terms used herein are believed to be well understood by one of ordinary skill in the art, definitions are set forth to facilitate explanation of the presently disclosed subject matter.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims.

Unless otherwise indicated, all numbers expressing quantities, properties, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about”, when referring to a value or to an amount of mass, weight, time, volume, concentration or percentage is meant to encompass variations of in some embodiments +/−50%, in some embodiments +/−40%, in some embodiments +/−30%, in some embodiments +/−20%, in some embodiments +/−10%, in some embodiments +/−5%, in some embodiments +/−1%, in some embodiments +/−0.5%, and in some embodiments +/−0.1% from the specified amount, as such variations are appropriate to perform the disclosed method.

Alternatively, the terms “about” or “approximately” can mean within an acceptable error range for the particular value as determined by one of ordinary skill in the art, which will depend in part on how the value is measured or determined, i.e., the limitations of the measurement system. For example, “about” can mean within 3, or more than 3, standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise indicated, all numbers expressing quantities, properties, and so forth used in the specification and claims are to be understood as being modified in all instances by the term “about”. And so, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

### Proposed Sensing Mechanism

In some embodiments, the methods and systems described herein can comprise a new test assay to detect cationic polymers, wherein sodium citrate is a ligand that can be used to stabilize the colloidal AuNPs. This stabilization can arise because of the strong electrostatic repulsion between negatively charged citrate ions on the surfaces of different AuNPs, thereby preventing AuNP aggregation.

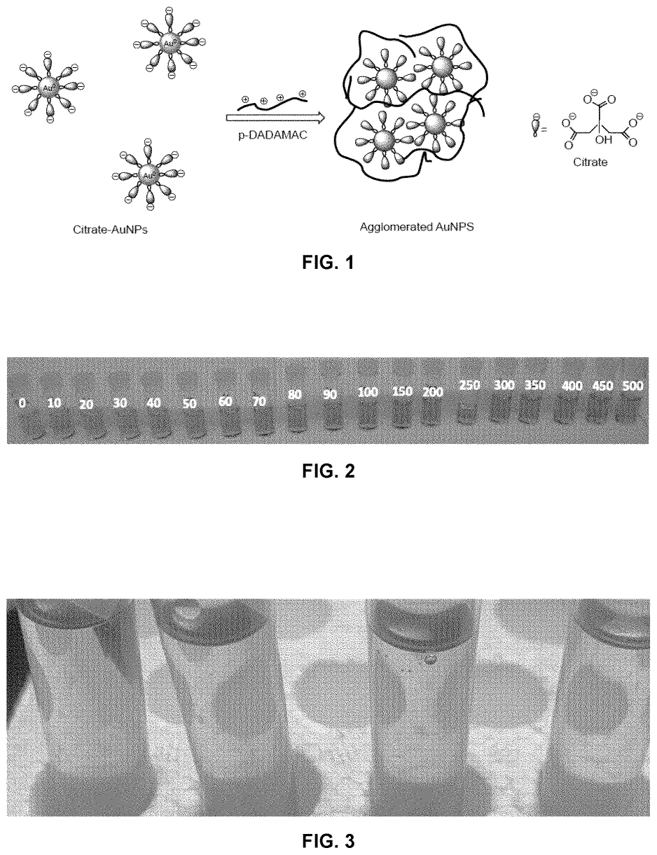

In the presence of positively charged cationic polymers, the polymer can bind to the cit-AUNPs, passivating their charge and then the cit-AuNPs aggregate can result in the appearance of a detectable colour change, as shown in FIG. 1. In some embodiments, anionic polymer detection can occur using a similar mechanism, with the charge on the surface of the gold and the polymer reversed.

### Optimization of the Sensing System

In some embodiments, the parameters for the assay can ensure that a colour change can occur at approximately 150 μg/L of cationic polymer in distilled water. By diluting the analyte sample to various dilutions using test solutions and testing empirically, the dilution factor can be used to estimate the cationic polymer.

At a cationic polymer concentration of 30 μg/L, cit-AuNP aggregation can be negligible, and the solution appears red as shown in FIG. 2. When increasing the cationic polymer concentration in the range of 40-100 μg/L, the aggregation increased and can manifest in a visual colour change as shown in FIG. 2. At 150 μg/L of cationic polymer, the colour can change from purple to blue as shown in FIG. 2. At higher cationic polymer concentrations (i.e., >150 μg/L), the absorbance ratio can plateau. These results indicate that higher concentrations of cationic polymer can induce a higher degree of aggregation of the cit-AuNPs and that at very high concentrations, few, if any, free cit-AuNPs are present.

### Interfering Environmental Factors

To test the impact of common environmental interferents on the cit-AuNP aggregation, UV-vis spectra were acquired for mixtures containing key metal ions (i.e., Ca²⁺, Mg²⁺, Mn²⁺, Hg²⁺, Fe³⁺, Zn²⁺, Cu²⁺, Pb²⁺, and Cr³⁺) at 1, 5, and 10 μM. At these concentrations, the metal ions tested did not influence the cit-AuNP spectra. However, at environmentally relevant concentrations (i.e., 165-216 mg/L), Ca²⁺ and Mg²⁺ ions generated significant interference (i.e., AuNP aggregation) that prevented accurate detection of p-DADMAC. Therefore, a complexing chelating agent, ethylenediaminetetraacetic acid (“EDTA”), can be employed in advance for all water samples to remove the complicating effects of Ca²⁺ and Mg²⁺ ions.

Dissolved organic carbon (“DOC”) likewise, can affect the colour development of the assay, and dilution of test samples with distilled water combined with EDTA can be used to ensure clear results. Comparison of test results with reference solutions is required by the assay.

### Example

Referring to FIG. 3, illustrates assay colour development in p-DADMAC reference solutions comprising river water (upstream of polymer addition site and, therefore, free of polymers) containing metals and DOC (instead of distilled water).

If the cationic polymer concentration in test sample is <<200 ug/L, a full-strength analysis of the test sample can remain orange/red in colour. This cationic polymer concentration is below the hazard concentration for cationic polymers (~300 ug/L).

If the [cationic polymer] in test solution is ≥1000 ug/L, the then test sample must be diluted with a diluent and the dilution factor noted.

If the test sample is diluted 10× (9 parts diluent and 1 part test sample), and the colour turns blue, then the [cationic polymer] is ≥1 mg/L (200 μg/L×10-fold dilution factor) and the sample must be further diluted, and dilution factor noted when test solution does not change colour.

If, however, the colour remains pink/red after dilution, the cationic polymer is <200 μg/L. More precise determinations can be achieved by varying the dilution factor.

In some embodiments, the present sensor system can detect cationic polymer at very low concentrations. To do so, it is necessary to minimize the ‘red’ background arising from ‘free’ cit-AuNPs while maximizing the intensity of the aggregate absorbance.

In some embodiments, modified AuNPs can be employed as a simple plasmonic sensor for colorimetric determination of cationic polymer in aqueous medium based on aggregation of cit-AuNPs. The presence of cationic polymer can induce obvious aggregation of cit-AuNP and can provide a visual colorimetric for detection of cationic polymer by monitoring a colour change from red to purple/blue. The methods and systems described herein can provide high sensitivity and selectivity with short analysis time and low cost.

In some embodiments, a system for colorimetric determination of cationic polymer in aqueous medium based on aggregation of cit-AuNPs can comprise:
Standard (“STD”) high (Standard P800™ 1 mg/L)¹
STD low (Standard P800™ 0.2 mg/L)²
Solution A (EDTA 0.1M)
Solution B (AuNP@cit 0.18 mg/mL)
Distilled water
1 ml syringe for polymer water samples
1 ml syringe for polymer-free water samples (blanks)
1 mL syringe for distilled water
1 ml syringe for Solution A (EDTA 0.1M)
   ¹ A poly-DADMAC available from Clearflow Group Inc. of Sherwood Park, Alberta, Canada. ² lbid.

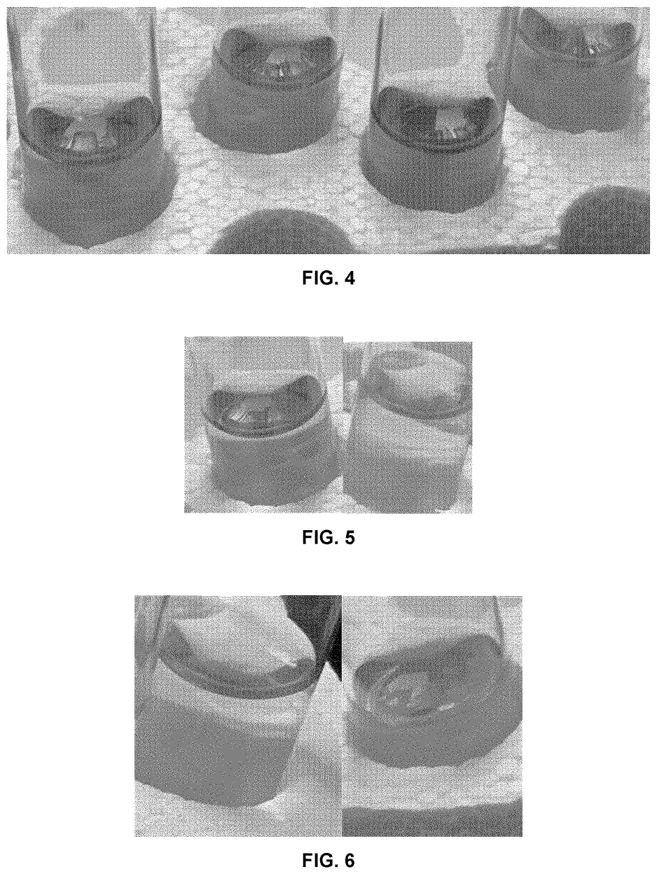

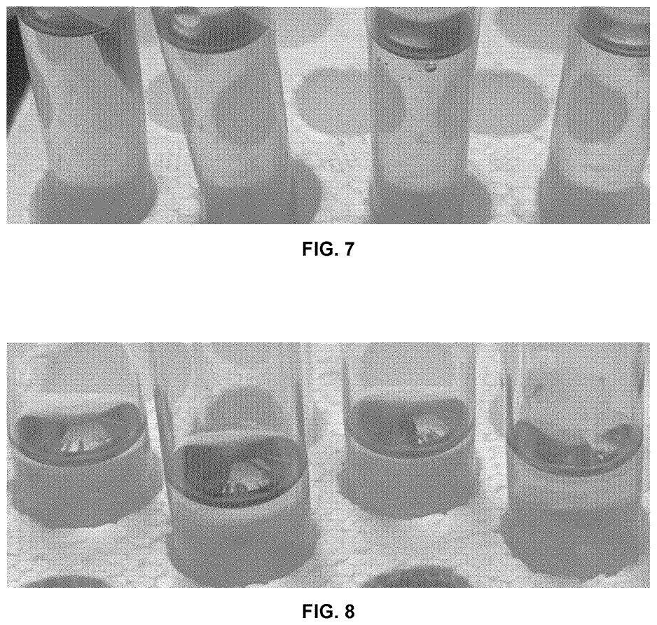

In some embodiments, the method for colorimetric determination of cationic polymer in aqueous medium based on aggregation of cit-AuNPs can comprise:
1. Make up high-concentration or first reference solution having a first colour (1 mg/L) (FIGS. 4 and 7):
   1.1. Using 1 ml syringe add 0.75 mL of Solution A (EDTA 0.1M) to 7 mL glass vial 1
   1.2. Using 3 ml syringe add 2 mL of STD high to vial 1
   1.3. Using 3 ml syringe add 2 mL of distilled water to vial 1
   1.4. Cap vial 1 and shake for 1 min
   1.5. Uncap vial 1 and using 1 ml syringe add 0.25 mL of Solution B (AuNP@cit 0.18 mg/mL)
   1.6. Cap vial 1 shake for a few seconds ; allow 10 minutes for full colour development (the colour will last for at least 24 hrs at room temperature)

The first colour should be bluish or bluish purple (dissolved organic carbon concentrations in water affect the final colour development).
2. Make up medium-concentration or second reference solution having a second colour (0.5 mg/L) (FIG. 7):
   2.1. Using 1 ml syringe add 0.75 mL of Solution A (EDTA 0.1M) to 7 mL glass vial 2
   2.2. Using 3 ml syringe add 1 mL of STD high to vial 2
   2.3. Using 3 ml syringe add 3 mL of distilled water to vial 2
   2.4. Cap vial 2 and shake for 1 min
   2.5. Uncap vial 2 and using 1 ml syringe add 0.25 mL of Solution B (AuNP@cit 0.18 mg/mL)
   2.6. Cap vial 2 shake for a few seconds ; allow 10 minutes for full colour development (the colour will last for at least 24 hrs at room temperature)

The second colour should be bluish purple/pinkish purple (dissolved organic carbon concentrations in water affect the final colour development).
3. Make up low concentration or third reference solution having a third colour (0.2 mg/L) (FIGS. 5 and 7):
   3.1. Using 1 mL syringe add 0.75 mL of Solution A (EDTA 0.1M) to 7 mL glass vial 3
   3.2. Using 3 ml syringe add 2 mL of STD low to vial 3
   3.3. Using 3 ml syringe add 2 mL of distilled water to vial 3
   3.4. Cap vial 3 and shake for 1 min
   3.5. Uncap vial 3 and using 1 mL syringe add 0.25 mL of Solution B (AuNP@cit 0.18 mg/mL)
   3.6. Cap vial 3 shake for a few seconds ; allow 10 minutes for full colour development (the colour will last for at least 24 hrs at room temperature)

The third colour should be a shade of pink/purplish pink-colour variations occur due to dissolved organic carbon concentrations in water.
4. Make up blank or control reference solution having a fourth colour (FIGS. 3 and 4):
   4.1. Using 1 ml syringe add 0.75 mL of Solution A (EDTA 0.1M) to 7 mL glass vial 4
   4.2. Using 3 mL syringe add 2 mL of polymer-free water (upstream of where the polymer is added in the field) to vial 4
   4.3. Using 3 ml syringe add 2 mL of distilled water to vial 4
   4.4. Cap vial 4 and shake for 1 min
   4.5. Uncap vial 4 and using 1 mL syringe add 0.25 mL of Solution B (AuNP@cit 0.18 mg/mL)
   4.6. Cap vial 4 shake for a few seconds; allow 10 minutes for full colour development (the colour will last for at least 24 hrs at room temperature)

The fourth colour should be a shade of pink-colour variations occur due to dissolved organic carbon concentrations in water.
5. Prepare field sample solution having a sample solution colour:
   5.1. Using 1 mL syringe add 0.75 mL of Solution A (EDTA 0.1M) to 7 mL glass vial 5
   5.2. Using 3 ml syringe add 2 mL of potentially polymer-containing water sample to vial 5
   5.3. Using 3 ml syringe add 2 mL of distilled water to vial 5
   5.4. Cap vial 5 and shake for 1 min
   5.5. Uncap vial 5 and using 1 ml syringe add 0.25 mL of Solution B (AuNP@cit 0.18 mg/mL)
   5.6. Cap vial 5 shake for a few seconds; allow 10 minutes for full colour development (the colour will last for at least 24 hrs at room temperature)

Compare the colour of the field sample solution to the first, second, third and fourth colours of the polymer reference solutions: high (vial 1), medium (vial 2), low concentration (vial 3) and blank (vial 4).

If the field sample solution colour matches the colour of the blank or control solution, ie. pink in colour, this indicates that polymer levels in the field sample solution are negligible. If the field sample solution displays purple and blue shades, this indicates that the field sample solution contains toxic concentrations of polymer.

### Precise Determination of Polymer Concentration in Field Sample

To determine the approximate concentration of the polymer in the field sample that turned blue, start with testing a one in ten dilution (1:10) of the original sample (take 1 mL of the field sample and add 9 mL of distilled water) then repeat the procedure IV above. If the sample remains blue, repeat with 1 in 20 dilution (1 mL of the original sample +19 mL of distilled water). Higher or lower dilution will be necessary until the diluted sample turns pinkish purple. Compare the colour to the reference solutions and then re calculate the original polymer concentration by multiplying the matching standard concentration by the dilution factor. As shown in FIG. 8, the blue and purple/pinkish purple results indicate toxic concentrations of polymer.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the embodiments described herein.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc. can be passed, forwarded, or transmitted via any suitable means including memory sharing, message passing, token passing, network transmission, etc.

The actual software code or specialized control hardware used to implement these systems and methods is not limiting of the embodiments described herein. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

We claim:

1. A method for detecting the concentration of cationic or anionic polymer in an aqueous solution where the cationic or anionic polymer has been introduced into the aqueous solution, the method comprising:
a) preparing a first reference solution having a first colour;
b) preparing a second reference solution having a second colour;
c) preparing a third reference solution having a third colour;
d) preparing a control reference solution having a fourth colour;
e) preparing a field sample solution having a sample solution colour; and
f) comparing the sample solution colour of the field sample solution to the first, second, third and fourth colours of the first, second, third and control reference solutions to determine the concentration of the cationic or anionic polymers in the field sample solution.

2. The method as set forth in claim 1, wherein preparing the first reference solution comprises:
a) adding a chelating agent to a first vial;
b) adding a first reference polymer solution to the first vial, the first reference polymer solution comprising a first concentration;
c) adding distilled water to the first vial;
d) mixing the chelating agent and the first reference polymer solution with the distilled water in the first vial;
e) adding a reference citrate-capped gold nanoparticle (AuNP) solution to the first vial; and
f) mixing the AuNP solution with the chelating agent and the first reference polymer solution with the distilled water in the first vial thereby producing the first reference solution having the first colour.

3. The method as set forth in claim 2, wherein the chelating agent comprises ethylenediaminetetraacetic acid.

4. The method as set forth in claim 3, wherein the chelating agent comprises a concentration of about 0.1 moles/litre.

5. The method as set forth in claim 2, wherein the first reference polymer solution comprises a concentration of about 1 milligram/litre.

6. The method as set forth in claim 2, wherein the AuNP solution comprises a concentration of about 0.18 milligram/millilitre.

7. The method as set forth in claim 1, wherein preparing the second reference solution comprises:
a) adding a chelating agent to a second vial;
b) adding a first reference polymer solution to the second vial, the first reference polymer solution comprising a first concentration;
c) adding distilled water to the second vial;
d) mixing the chelating agent and the first reference polymer solution with the distilled water in the second vial;
e) adding a reference citrate-capped gold nanoparticle (AuNP) solution to the second vial; and
f) mixing the AuNP solution with the chelating agent and the first reference polymer solution with the distilled water in the second vial thereby producing the second reference solution having the second colour.

8. The method as set forth in claim 7, wherein the chelating agent comprises ethylenediaminetetraacetic acid.

9. The method as set forth in claim 8, wherein the chelating agent comprises a concentration of about 0.1 moles/litre.

10. The method as set forth in claim 7, wherein the first reference polymer solution comprises a concentration of about 1 milligram/litre.

11. The method as set forth in claim 7, wherein the AuNP solution comprises a concentration of about 0.18 milligram/millilitre.

12. The method as set forth in claim 1, wherein preparing the third reference solution comprises:
a) adding a chelating agent to a third vial;
b) adding a second reference polymer solution to the third vial, the second reference polymer solution comprising a second concentration;
c) adding distilled water to the third vial;
d) mixing the chelating agent and the second reference polymer solution with the distilled water in the third vial;
e) adding a reference citrate-capped gold nanoparticle (AuNP) solution to the third vial; and
f) mixing the AuNP solution with the chelating agent and the second reference polymer solution with the distilled water in the third vial thereby producing the third reference solution having the third colour.

13. The method as set forth in claim 12, wherein the chelating agent comprises ethylenediaminetetraacetic acid.

14. The method as set forth in claim 13, wherein the chelating agent comprises a concentration of about 0.1 moles/litre.

15. The method as set forth in claim 12, wherein the second reference polymer solution comprises a concentration of about 0.2 milligram/litre.

16. The method as set forth in claim 12, wherein the AuNP solution comprises a concentration of about 0.18 milligram/millilitre.

17. The method as set forth in claim 1, wherein preparing the control reference solution comprises:
a) adding a chelating agent to a fourth vial;
b) adding a polymer-free sample of the aqueous solution to the fourth vial;
c) adding distilled water to the fourth vial;
d) mixing the chelating agent and the polymer-free sample of the aqueous solution with the distilled water in the fourth vial;
e) adding a reference citrate-capped gold nanoparticle (AuNP) solution to the fourth vial; and
f) mixing the AuNP solution with the chelating agent and the polymer-free sample of the aqueous solution with the distilled water in the fourth vial thereby producing the control reference solution having the fourth colour.

18. The method as set forth in claim 17, wherein the chelating agent comprises ethylenediaminetetraacetic acid.

19. The method as set forth in claim 18, wherein the chelating agent comprises a concentration of about 0.1 moles/litre.

20. The method as set forth in claim 17, wherein the polymer-free sample is drawn from a location upstream from where the cationic or anionic polymer has been introduced into the aqueous solution.

21. The method as set forth in claim 17, wherein the AuNP solution comprises a concentration of about 0.18 milligram/millilitre.

22. A system for detecting the concentration of cationic or anionic polymer in an aqueous solution where the cationic or anionic polymer has been introduced into the aqueous solution, the system comprising:
a) a first reference polymer solution;
b) a second reference polymer solution;
c) a chelating agent;
d) a reference citrate-capped gold nanoparticle (AuNP) solution; and
e) distilled water.

23. The system as set forth in claim 22, further comprising a plurality of vials.

24. The system as set forth in claim 23, further comprising a plurality of syringes for transferring fluids into the vials.

25. The system as set forth in claim 22, wherein the first reference polymer solution comprises a concentration of about 1 milligram/litre.

26. The system as set forth in claim 22, wherein the second reference polymer solution comprises a concentration of about 0.2 milligram/litre.

27. The system as set forth in claim 22, wherein the chelating agent comprises ethylenediaminetetraacetic acid.

28. The system as set forth in claim 27, wherein the chelating agent comprises a concentration of about 0.1 moles/litre.
